(19) United States
(12) Plant Patent Application Publication
(10) Pub. No.: US 2016/0324058 P1
(43) Pub. Date: Nov. 3, 2016

(54) Delphinium plant named 'Coadelfla'

(50) ;

(71) Applicant: Anthony Paul Coakley, Glasgow (GB)

(72) Inventor: Anthony Paul Coakley, Glasgow (GB)

(21) Appl. No.: 14/545,380

(22) Filed: Apr. 28, 2015

(51) Int. Cl. A01H 5/00

(52) U.S. Cl. PLT/423

(57) ABSTRACT

‘Coadelfla’ is a distinctive variety of Delphinium which is characterized by the combination of flowers with a large quantity of tepals and petaloids that exhibit a unique color combination of light shades of red-purple on outer tepal whorls to dark shades of red-purple on inner tepal whorls, an upright plant habit, a vigorous rate of growth, and the stability and uniformity of these traits through successive cycles of asexual propagation. The new cultivar produces a strong main flowering stem as well as a free and continuous bloom habit from early through late summer.

### LATIN NAME OF THE GENUS AND SPECIES

The Latin name of the genus and species of the novel variety disclosed herein is Delphinium hybrid.

### VARIETY DENOMINATION

The inventive variety of Delphinium disclosed herein has been given the variety denomination ‘Coadelfla’.

### BACKGROUND OF THE INVENTION

The present invention relates to a new and distinct cultivar of Delphinium, which has been given the variety denomination of ‘Coadelfla’. Its market class is PLT/423. The genus Delphinium is commonly known as a member of the Ranunculaceae family and may also be referred to as Larkspur. Delphinium plants bear inflorescences of a showy spike or racemes that may sometimes be paniculate and contain few to many flowers on erect and sometimes branching stems. The genus Delphinium is unusual in that all three primary colors are represented in different species. The tall hybrids with their long spikes are available in a wide range of colors including purple, mauve, pink, white, and cream as well as varying shades of blue. In Europe, they are grown as perennials but are treated as annuals in California and regions of similar climate.

### Parentage

The Delphinium variety ‘Coadelfla’ is the result of a planned breeding program carried out in Glasgow, Scotland. ‘Coadelfla’ is a seedling selection which resulted from the controlled pollination of two proprietary Delphinium hybrid breeding accessions (unnamed, uncommercialized and undistributed) developed and owned by the inventor. The initial selection of the cultivar now known as ‘Coadelfla’ was made in August of 2008. At that time it was determined that said cultivar exhibited certain distinguishing characteristics that would prove favorable for commercial production and marketability.

### Asexual Reproduction

‘Coadelfla’ was first asexually propagated by tissue culture in 2012 at a tissue culture laboratory in Roelofarendsveen, The Netherlands under the control of the inventor. The distinctive characteristics of the inventive ‘Coadelfla’ cultivar are stable from generation to generation; clones of the cultivar produced by tissue culture maintain the distinguishing characteristics of the original plant.

### SUMMARY OF THE INVENTION

The following characteristics have been repeatedly observed and represent the distinguishing characteristics of the new Delphinium cultivar ‘Coadelfla’. These traits in combination distinguish ‘Coadelfla’ as a new and distinct cultivar.
1. Delphinium ‘Coadelfla’ exhibits a floret with a high tepal and petaloid count.
2. Delphinium ‘Coadelfla’ exhibits a unique color combination within the floret.
3. Delphinium ‘Coadelfla’ exhibits an upright growth habit.
4. Delphinium ‘Coadelfla’ exhibits a high degree of plant vigor.
5. Delphinium ‘Coadelfla’ exhibits a primary inflorescence with a strong peduncle.
6. Delphinium ‘Coadelfla’ exhibits a freely and continuous bloom habit.

### BRIEF DESCRIPTION OF THE FIGURES

FIG. 1 shows, as nearly true as it is reasonably possible to make the same in color illustrations of this type, the foliage of ‘Coadelfla’. The foliage shown was removed from a plant grown outdoors in Glasgow, Scotland using conventional production practices typically employed for producing Delphinium plants. No disease or pest measures were taken in producing this plant.

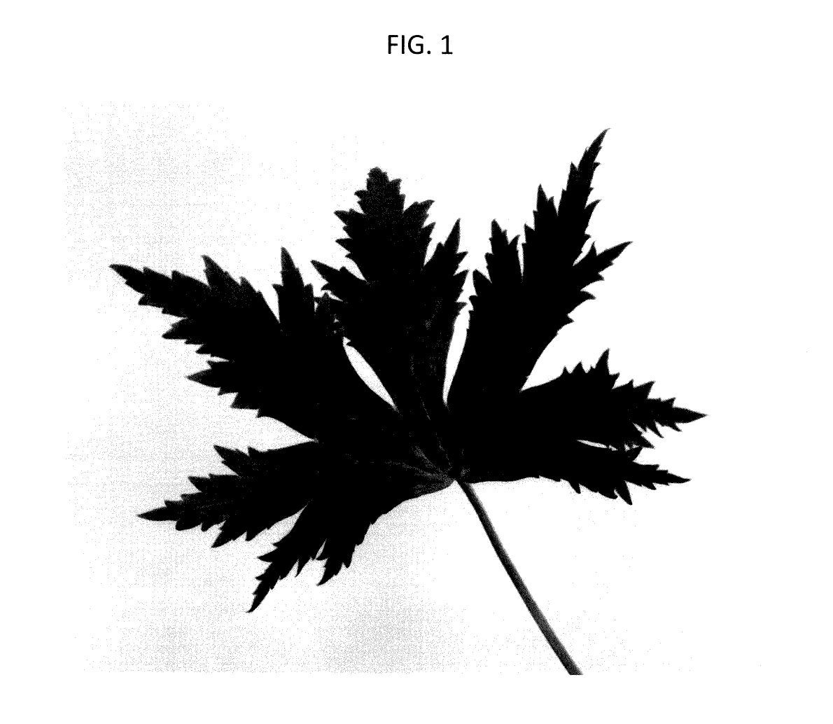

FIG. 2 shows, as nearly true as it is reasonably possible to make the same in color illustrations of this type, the inflorescence of ‘Coadelfla’. The inflorescence shown was removed from a plant grown outdoors in Glasgow, Scotland using conventional production practices typically employed for producing Delphinium plants. No disease or pest measures were taken in producing this plant.

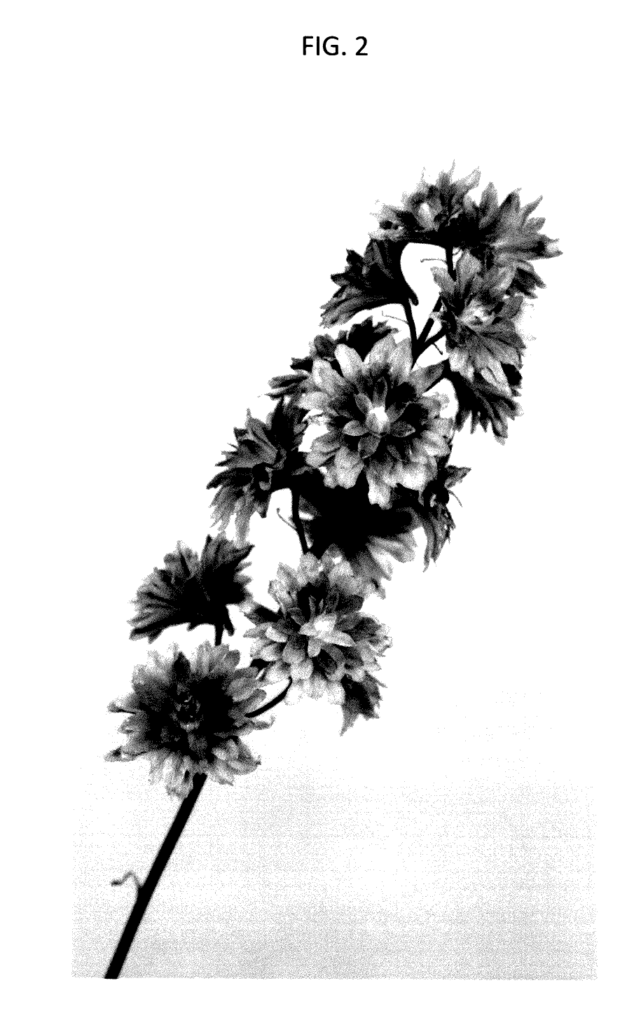

### BOTANICAL DESCRIPTION OF THE PLANT

The following is a detailed botanical description of a new and distinct variety of Delphinium known as ‘Coadelfla’, based upon observations of a 9 month old plant grown outdoors in Glasgow, Scotland in a one liter nursery pot from winter to summer, using conventional production practices typically employed for producing Delphinium plants. No disease or pest measures were taken in producing this plant. Observation data was recorded in August of 2014.

Those skilled in the art will appreciate that certain characteristics will vary with older or, conversely, with younger plants. ‘Coadelfla’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, 2007 edition.

A botanical description of ‘Coadelfla’ and comparisons with other varieties of Delphinium are provided below.

### Technical Description of the Variety

Plant description:
   Growth rate.—Moderate.
   Growth habit.—Upright perennial flowering plant with flowers arranged on terminal racemes; numerous racemes per plant.
   Height.—Approximately 89.0 cm.
   Width.—Approximately 46.0 cm.
   Environmental tolerances.—Tolerates temperatures as low as 5 approximately minus 20 degrees Celsius (twenty degrees below freezing); upper temperature tolerance is unknown. No tolerance for drought.
   Disease and pest resistance.—Plants of the claimed plant have not been noted to be resistant to pathogens and pests common to Delphinium.
   Propagation.—Tissue culture.
   Time to produce a rooted cutting.—Approximately 4 to 5 weeks to produce a rooted cutting.
Root system: Fine, fibrous; freely branched and moderately dense.
Stem:
   Primary stems.—Four, as observed.
   Stem length.—Approximately 32.7 cm, excluding the inflorescence.
   Stem width.—Approximately 1.9 cm.
   Stem color.—Near RHS 146A to 146B.
   Stem texture.—Matte, sparsely to moderately pubescent with short soft hairs of an average length of 1 mm and a color near RHS NN155D.
   Internode length.—Approximately 0.8 cm.
Foliage:
   Leaf arrangement.—Alternate.
   Leaf attachment.—Petiolate.
   Quantity of leaves per plant.—Approximately 41 per stem.
   Leaf length.—Average of 15.3 cm in length.
   Leaf width.—Average of 20.5 cm in width.
   Leaf shape.—Pedately lobed.
   Leaf apex.—Acute.
   Leaf base.—Hastate.
   Leaf margin.—Biserrate.
   Texture (adaxial surface).—Glabrous; matte.
   Pubescence (adaxial surface).—Absent.
   Young leaf color (adaxial surface).—Near RHS 143B.
   Young leaf color (abaxial surface).—In between near RHS 138B and 143C.
   Mature leaf color (adaxial surface).—Near RHS N137B.
   Mature leaf color (abaxial surface).—Near RHS 147B.
   Venation.—Laciniate.
   Vein color (adaxial surface).—Near RHS 144B.
   Vein color (abaxial surface).—Near RHS 144A.
   Petiole.—Approximately 12.1 cm long and 0.35 cm wide; color is near 144A.
Inflorescence:
   Inflorescence type.—Terminal raceme.
   Inflorescence dimensions.—Approximately 57.1 cm long, as measured from the lowest flower to the apex of the inflorescence, and 11.9 cm wide. Bracts — Quantity — Two bracts are present at the base of each pedicel. Shape — Linear. Length — 0.9 cm, on average. Width — 0.8 mm, on average. Apex — Narrowly acute. Margin — Entire. Color (adaxial and abaxial surfaces) — Near RHS 152C.
Bud:
   Flower bud shape.—Obovate.
   Flower bud length.—1.8 cm, excluding the spur.
   Flower bud diameter.—Averaging 1.2 cm.
   Bud color (adaxial surface).—Near RHS N77D.
   Bud color (abaxial surface).—Near RHS 149D; slightly lighter coloration.
Flower:
   Flowering quantity.—About 160 buds and 44 fully opened flowers per lateral branch, at one time.
   Natural flowering season.—Midsummer to late summer.
   Flower longevity on plant.—Approximately three weeks.
   Pedicel.—Length — Approximately 5.2 cm. Diameter — Approximately 0.15 cm. Texture — Smooth; glabrous. Color — Near RHS 143A and 147B, tinged N186C.
   Perianth.—Consisting of a spur, a single whorl of tepals and multiple whorls petaloids; the approximate diameter is 5.0 cm and the approximate depth is 2.7 cm, excluding the spur. Aspect of corolla is upward to slightly pendulous. Flowers are persistent. Tepal Description — Arrangement — Rotate. Quantity — Five. Length — Approximately 2.6 cm. Width — Two lower tepals average 0.9 cm and three upper tepals average 1.2 cm. Shape — Narrowly obovate. Margin — Entire, undulate; sparsely covered with very thin short hairs with an average length of 0.1 cm; color is near RHS N155D. Apex — Broad acute. Texture — Glabrous, slightly glossy and slightly velvety. Aspect — Slightly curved backwards. Color, When Opening (adaxial surface) — Near RHS 76A; the base and also a darker localized area or “dot” at the apex with color near RHS 146A to 146B. Color, When Opening (abaxial surface) — Slightly lighter than RHS N77D; the base and also a darker localized area or “dot” at the apex with color near RHS 146A. Color, Fully Opened (adaxial surface) — Near RHS 76A; the base and also a darker localized area or “dot” at the apex with color near RHS 146A to 146B. Color, Fully Opened (abaxial surface) — Slightly lighter than RHS N77D; the base and also a darker localized area or “dot” at the apex with color near RHS 146A. Petaloid Description — Quantity — 48. Arrangement — Rotate. Length — Average: 2.4 cm. Width — Average: 0.7 cm. Shape — Narrowly obovate to oblong. Margin — Entire, undulate; sparsely covered with very thin short hairs with an average length of 0.1 cm; color is near RHS N155D. Apex — Broad acute. Texture — Glabrous, slightly glossy, and slightly velvety. Aspect — Very slightly curved backwards. Color, When Opening (adaxial surface) — Near RHS N77B. Color, When Opening (abaxial surface) — Slightly lighter than RHS N77D; the base and also a darker localized area or “dot” at the apex with color near RHS 146A. Color, Fully Opened (adaxial surface) — Near RHS 76A; the base and also a darker localized area or “dot” at the apex with color near RHS 146A to 146B. Color, Fully Opened (abaxial surface) — Slightly lighter than RHS N77D; the base and also a darker localized area or “dot” at the apex with color near RHS 146A.
   Spur.—Shape — Oblong; narrow. Quantity per flower — 1. Length — Approximately 0.8 cm. Width — Approximately 0.25 cm. Margin — Entire. Texture — Glabrous, bullate. Color — Near RHS N77D.
Reproductive organs:
   Stamens.—No stamens are present; all stamens are transformed into petaloids.
   Pistil.—Pistil number — Average of 3. Pistil length, Including Style — Approximately 0.2 cm. Style — Length — Approximately 1.75 mm. Color — Near RHS NN155D. Stigma — Shape — Pointed. Color — Near RHS NN155D. Ovary color — Near RHS 150B.
Seed and fruit: None observed.

### Comparisons With The Parents

Plants of the new cultivar ‘Coadelfla’ are similar to plants of the seed parent variety, an unnamed Delphinium hybrid, in most horticultural characteristics. However, flowers of the new variety have a higher tepal count with a stronger, unique and more consistent color.

Plants of the new cultivar ‘Coadelfla’ are similar to plants of the pollen parent variety, an unnamed Delphinium hybrid, in most horticultural characteristics. However, flowers of the new variety have a higher tepal count with a stronger, unique and more consistent color.

### Comparisons With Similar Delphinium Cultivars Known to The Inventor

The inventor is unaware of similar commercial varieties for comparison to ‘Coadelfla’. The strong flowering stems, unique flower coloration and increased is tepal count make the new cultivar unlike other Delphiniums in the trade.

That which is claimed is:

1. A new and distinct variety of Delphinium plant named ‘Coadelfla’, substantially as described and illustrated herein.
